(19) United States
(12) Patent Application Publication
(10) Pub. No.: US 2009/0010982 A1
(43) Pub. Date: Jan. 8, 2009

(54) BIOCOMPATIBLE ADHERENT SHEET FOR TISSUE SEALING

(75) Inventors: John M. Abrahams, Scarsdale, NY (US); Weiliam Chen, Mount Sinai, NY (US)

Correspondence Address: SCHWEGMAN, LUNDBERG & WOESSNER, P.A., P.O. BOX 2938, MINNEAPOLIS, MN, 55402, US

(73) Assignee: Endomedix, Inc., scarsdale, NY (US)

(21) Appl. No.: 12/136,182

(22) Filed: Jun. 10, 2008

Related U.S. Application Data
(63) Continuation-in-part of application No. 11/530,362, filed on Sep. 8, 2006.
Application No. 11/530,362 is a continuation-in-part of application No. 11/379,182, filed on Apr. 18, 2006.

(51) Int. Cl. A61L 27/54; A61L 24/08; A61L 27/26

(52) U.S. Cl. 424/422; 523/118

(57) ABSTRACT

A biocompatible adherent sheet for use in surgical and medical procedures for sealing the tissues of a living mammal is provided. The biocompatible adherent sheet includes a carrier sheet including a biocompatible polymer and a modified chitosan evenly disposed on one or both sides of the carrier sheet. Methods of preparing a biocompatible adherent sheet and methods of using a biocompatible adherent sheet are also provided. The biocompatible adherent sheet may also include a bioactive agent.

### CLAIM OF PRIORITY FROM A PRIOR-FILED APPLICATION

This application is a continuation-in-part of U.S. application Ser. No. 11/530,362, filed Sep. 8, 2006, which is a continuation-in-part of U.S. application Ser. No. 11/379,182, filed Apr. 18, 2006, both of which are incorporated herein by reference in their entirety, and claims the priority thereof.

### FIELD OF THE INVENTION

The invention relates to biocompatible adherent sheets for application to body surfaces for medical and veterinary use, methods of preparing the biocompatible adherent sheets, and methods of using the biocompatible adherent sheets.

### BACKGROUND OF THE INVENTION

Tissue sealants are increasingly important adjuncts in surgical procedures, being used in fields such as vascular surgery, cardiac surgery, spine surgery, and brain surgery as well as in general surgery. Uses for tissue sealants include, among others, augmenting or replacing sutures to join tissues or place them in proximity, closing perforations in biological membranes to prevent leakage of fluids, incorporating medicinal substances at the location of emplacement for localized release, and filling areas of tissue removal.

One commonly used tissue sealant is fibrin glue, a material analogous to clotted blood, which is obtained from reaction of fibrinogen and thrombin isolated from blood plasma. Commercial fibrin glues consist of a highly concentrated fibrinogen solution to be mixed with a thrombin solution before application to the surgical wound. These two-component fibrin glues are valuable in various surgical procedures, but may be washed away before hemostasis is achieved if the bleeding is heavy. The two-component fibrin glues also need some preparatory steps, which include thawing or dissolution. Thus, they are rather impractical and cumbersome.

During the past few years numerous fibrin sealants became the methods of choice in various surgeries. However, in the majority of trials with fibrin glues, a collagen fleece was additionally used to improve hemostatic and adhesive features, indicating their disadvantages and their restrained use by the surgeons. One drawback of the fibrin glues has been that in case of major bleeding the glue is typically washed away before sufficient polymerization of fibrin has occurred.

Accordingly, there is a need in the art for a biocompatible tissue sealant that is easy to use and overcomes the problems of the existing materials.

### SUMMARY OF THE INVENTION

The present invention provides a biocompatible adherent sheet for use in surgical and medical procedures for sealing the tissues of a living mammal, preferably a human. The biocompatible adherent sheet includes a carrier sheet including a biocompatible polymer and a modified chitosan evenly disposed on one or both surfaces of the carrier sheet. Methods of preparing a biocompatible adherent sheet and methods of using a biocompatible adherent sheet are also provided. The biocompatible adherent sheet may also include a bioactive agent and other active ingredients. The biocompatible adherent sheet is a soft, pliable material that adheres to various tissues, bends easily around curved surfaces, and can withstand moderate burst pressures. Further, the biocompatible adherent sheet described herein provides better adhesion to body tissues than existing materials. The tack of the biocompatible adherent sheet to various bodily tissues can be custom-tailored by adjusting the chemical composition of the modified chitosan. As such, a complete product range of various biocompatible adherent sheets with each product optimized for each tissue is possible.

The present invention also provides a biocompatible adherent sheet for application to one or more moist body tissue surfaces for obtaining hemostasis, tissue sealing, tissue gluing, and filling tissue voids. The biocompatible adherent sheet includes a carrier sheet including a biocompatible polymer, wherein the carrier sheet is flexible, conformable, and of substantially uniform thickness; and a modified chitosan substantially evenly disposed on one or both surfaces of the carrier sheet, wherein the modified chitosan includes an alkylated chitosan, the reaction product of an alkylated chitosan and an acidic polysaccharide, the reaction product of an alkylated chitosan and an oxidized polysaccharide, the reaction product of an alkylated chitosan, an acidic polysaccharide, and an oxidized polysaccharide, or the reaction product of an alkylated chitosan, an acidic polysaccharide, a carboxyl activating reagent, and a dehydrating reagent, wherein the alkylated chitosan includes an acrylated chitosan or a poly(oxyalkylene)chitosan.

In one embodiment, the biocompatible polymer includes hyaluronic acid, polyhydroxy acid, lactic acid, glycolic acid, hydroxybutanoic acid, cellulose, gelatin, collagen, or a combination thereof. Preferably, the biocompatible polymer is collagen.

In one embodiment, the modified chitosan is distributed on one surface of the carrier sheet. In another embodiment, the modified chitosan is distributed on both surfaces of the carrier sheet.

In another embodiment, the acrylated chitosan includes an N-acrylated chitosan.

In one embodiment, the poly(oxyalkylene)chitosan includes a poly(oxyethylene)chitosan.

In one embodiment, the acidic polysaccharide includes a hyaluronan or a carboxymethylcellulose. In another embodiment, the acidic polysaccharide includes a hyaluronan. In yet another embodiment, the acidic polysaccharide includes a carboxymethylcellulose.

In one embodiment, the oxidized polysaccharide includes an oxidized dextran, an oxidized starch, or an oxidized hyaluronan.

In one embodiment, the carboxyl activating reagent includes an N-hydroxy compound.

In one embodiment, the dehydrating reagent includes a carbodiimide.

In one embodiment, the moist body tissue surfaces are in a gastrointestinal system, parenchymal organs, a cardiovascular system, a thoracic system, a pulmonary system, an ear area, a nose area, a throat area, a dental area, a gynecological system, a urological system, a vascular system, a bone system, a neurological system, a lymphatic system, a dermal surface, a biliary system, or a combination thereof.

In one embodiment, the carrier sheet, the modified chitosan, or both the carrier sheet and the modified chitosan, further comprise a bioactive agent. Preferably, the bioactive agent includes a hormone, an immunomodulator, an immunosuppressant, an antibiotic, a cytostatic, a diuretic, a gastrointestinal agent, a cardiovascular agent, a neuropharmaceutical, a blood coagulation inducing agent, or a combination thereof.

The present invention also provides a method of preparing a biocompatible adherent sheet for application to one or more moist body tissue surfaces for obtaining hemostasis, tissue sealing, tissue gluing, and filling tissue voids. The method includes disposing a modified chitosan substantially evenly on one or both surfaces of a carrier sheet including a biocompatible polymer, wherein the carrier sheet is flexible, conformable, and of substantially uniform thickness, and wherein the modified chitosan includes an alkylated chitosan, the reaction product of an alkylated chitosan and an acidic polysaccharide, the reaction product of an alkylated chitosan and an oxidized polysaccharide, the reaction product of an alkylated chitosan, an acidic polysaccharide, and an oxidized polysaccharide, or the reaction product of an alkylated chitosan, an acidic polysaccharide, a carboxyl activating reagent, and a dehydrating reagent, wherein the alkylated chitosan includes an acrylated chitosan or a poly(oxyalkylene)chitosan.

The present invention further provides a method of preparing a biocompatible adherent sheet for application to one or more moist body tissue surfaces. The method includes casting an aqueous carrier solution including a biocompatible polymer; freezing the aqueous carrier solution to provide a frozen aqueous carrier solution; casting an aqueous modified chitosan solution on one or more surfaces of the frozen aqueous carrier solution; freezing the aqueous modified chitosan solution to provide a frozen aqueous chitosan solution on one or more surfaces of the frozen aqueous carrier solution; and lyophilizing the frozen aqueous modified chitosan solution on one or more surfaces of the frozen aqueous carrier solution, wherein the aqueous modified chitosan solution includes an acrylated chitosan or the reaction product of a poly(oxyalkylene)chitosan and an acidic polysaccharide.

The present invention also provides a method of preparing a biocompatible adherent sheet for application to one or more moist body tissue surfaces. The method includes contacting a carrier sheet including a biocompatible polymer with an aqueous modified chitosan solution, wherein the carrier sheet is flexible, conformable, and of substantially uniform thickness, wherein the contacting includes immersing, spraying, dipping, spin coating, bar coating, knife coating, extrusion coating, or direct coating; freezing the carrier sheet contacted with the aqueous modified chitosan solution; and lyophilizing the frozen carrier sheet contacted with the aqueous modified chitosan solution, wherein the aqueous modified chitosan solution includes an acrylated chitosan or the reaction product of a poly(oxyalkylene)chitosan and an acidic polysaccharide.

The present invention further provides a method for obtaining hemostasis, tissue sealing, tissue gluing, and filling tissue voids. The method includes applying to one or more moist body tissue surfaces a biocompatible adherent sheet including a carrier sheet including a biocompatible polymer, wherein the carrier sheet is flexible, conformable, and of substantially uniform thickness; and a modified chitosan substantially evenly disposed on one or both surfaces of the carrier sheet, wherein the modified chitosan includes an alkylated chitosan, the reaction product of an alkylated chitosan and an acidic polysaccharide, the reaction product of an alkylated chitosan and an oxidized polysaccharide, the reaction product of an alkylated chitosan, an acidic polysaccharide, and an oxidized polysaccharide, or the reaction product of an alkylated chitosan, an acidic polysaccharide, a carboxyl activating reagent, and a dehydrating reagent, wherein the alkylated chitosan includes an acrylated chitosan or a poly(oxyalkylene)chitosan. In the biocompatible adherent sheets described herein, the modified chitosan acts as an adhesive material to bind the biocompatible adherent sheet to the tissue.

The present invention also provides a biocompatible adherent sheet for application to one or more moist body tissue surfaces for obtaining hemostasis, tissue sealing, tissue gluing, and filling tissue voids. The biocompatible adherent sheet includes a carrier sheet comprising a biocompatible polymer and an optional modified chitosan, wherein the carrier sheet is flexible, conformable, and of substantially uniform thickness, wherein the modified chitosan includes an acrylated chitosan or the reaction product of a poly(oxyalkylene)chitosan and an acidic polysaccharide; and an optional modified chitosan substantially evenly disposed on one or both surfaces of the carrier sheet.

In one embodiment, the obtaining hemostasis, tissue sealing, and tissue gluing occurs during surgical interventions in a gastrointestinal system, parenchymal organs, a cardiovascular system, a thoracic system, a pulmonary system, an ear area, a nose area, a throat area, a dental area, a gynecological system, a urological system, a vascular system, a bone system, a neurological system, a lymphatic system, a dermal surface, a biliary system, or a combination thereof.

In one embodiment, the biocompatible adherent sheet has a thickness of about 0.01 mm to about 50 mm, preferably about 1 mm to about 10 mm.

In one embodiment, the modified chitosan is present in an amount of about 0.1 mg/cm² to about 100 mg/cm², preferably about 1 mg/cm² to about 5 mg/cm².

In one embodiment, the carrier sheet has a density of about 1 mg/cm³ to about 100 mg/cm³, preferably about 3 mg/cm³ to about 60 mg/cm³, more preferably about 5 mg/cm³ to about 30 mg/cm³.

The biocompatible adherent sheets are useful for application to one or more moist body tissue surfaces to obtain, for example, hemostasis, tissue sealing, and tissue gluing. Further, the biocompatible adherent sheets offer outstanding advantages of ease of use, biocompatibility and biodegradability, suitability for use in conjunction with other surgical procedures, strength, adhesivity, and versatility.

### DETAILED DESCRIPTION OF THE INVENTION

The present invention provides a biocompatible adherent sheet for application to one or more moist body tissue surfaces to obtain, for example, hemostasis, tissue sealing, and tissue gluing. The present invention also provides methods of preparing a biocompatible adherent sheet along with a method of use thereof.

The biocompatible adherent sheet may be used in a wide variety of medical and surgical applications. For example, the biocompatible adherent sheet may be used to seal to seal leaks in lung tissue, act as a hemostatic agent after resection of tissue, seal the dura mater, act as an anti-adhesion barrier, seal tissue planes, and provide delivery of drugs. Further advantages of the biocompatible adherent sheet may include, for example, (1) ready-to-use application, (2) easily applied directly onto tissue and organ surfaces, (3) good flexibility and conformability, (4) ability to withstand stretching and compression, (5) effective hemostasis and tissue sealing within a few minutes without runoff, (6) a favorable safety profile, and (8) custom-tailored biodegradability.

The biocompatible adherent sheet is useful for hemostasis, tissue gluing and tissue sealing, in particular in surgical intervention in the gastrointestinal system, such as the esophagus, stomach, small intestine, large intestine, rectum, on parenchymal organs, such as liver, spleen, pancreas, kidneys, lungs, adrenal glands, thyroid and lymph nodes, cardiovascular surgery, thoracic surgery including surgery on the trachea, bronchi or lungs, surgical interventions in the ear, nose and throat (ENT) area including dental surgery, gynecological, urological, bone (e.g., spongiosa resection), and emergency surgery, neurological surgery, lymphatic, biliary, and cerebrospinal (CSF) fistulae, and air leakages during thoracic and pulmonary surgery.

The biocompatible adherent sheets may be fabricated to be substantially air tight and liquid tight. These features make the biocompatible adherent sheets particularly useful to treat lymphatic, biliary, and cerebrospinal (CSF) fistulae, and air leakages during pulmonary and thoracic surgery. Further, due to the biocompatible adherent sheets being substantially liquid tight, they are highly useful, for example, in surgery of highly vascularized organs such as the liver and spleen, and for surgery in the gastrointestinal channel. The biocompatible adherent sheets are to be applied when bleeding, or lymphatic, biliary, air or CSF leakage cannot be controlled with conventional methods or when these methods would yield unfavorable results.

The biocompatible adherent sheets described herein have many advantages over existing fibrin glue-collagen technologies. For example, fibrin is derived from human plasma, expensive to manufacture, and has risks related to blood borne pathogens. In contrast, chitosan is hemostatic, antimicrobial, easy to manufacture, and easy to hybridize to existing biocompatible polymers. Further, chitosan degrades over a longer time span than fibrin glue. By modifying the chitosan and applying the modified chitosan to collagen, as described herein, the resulting biocompatible adherent sheet is adherent and typically degrades over two to three months instead of fourteen to twenty-one days, as is typical with the existing fibrin glue-collagen technologies.

While not being bound by theory, it is believed that the modified chitosan exhibits better adhesion than fibrin glue through several mechanisms. First, the surface of modified chitosan is very abundant in hydroxyl, amino, and carboxylic acids groups, all of which may form extensive hydrogen bonds with the body tissue. Second, the amino and carboxylic acid groups are capable of electrostatically interacting with opposite charges on the surface of the body tissue. Third, the modified chitosan is a linear flexible molecule, unlike branched macromolecules in fibrin glue that may become rigid.

Reference will now be made in detail to certain claims of the disclosed subject matter, examples of which are illustrated in the accompanying structures and formulas. While the disclosed subject matter will be described in conjunction with the enumerated claims, it will be understood that they are not intended to limit the disclosed subject matter to those claims. On the contrary, the disclosed subject matter is intended to cover all alternatives, modifications, and equivalents, which may be included within the scope of the presently disclosed subject matter as defined by the claims.

References in the specification to “one embodiment” indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

Unless otherwise indicated, the words and phrases presented in this document have their ordinary meanings to one of skill in the art. Such ordinary meanings can be obtained by reference to their use in the art and by reference to general and scientific dictionaries, for example, Webster's Third New International Dictionary, Merriam-Webster Inc., Springfield, Mass., 1993, The American Heritage Dictionary of the English Language, Houghton Mifflin, Boston Mass., 1981, and Hawley's Condensed Chemical Dictionary, 14^{th} edition, Wiley Europe, 2002.

The following explanations of certain terms are meant to be illustrative rather than exhaustive. These terms have their ordinary meanings given by usage in the art and in addition include the following explanations.

As used herein, the term “about” refers to a variation of 10 percent of the value specified; for example about 50 percent carries a variation from 45 to 55 percent.

As used herein, the term “and/or” refers to any one of the items, any combination of the items, or all of the items with which this term is associated.

As used herein, the singular forms “a,” “an,” and “the” include plural reference unless the context clearly dictates otherwise.

As used herein, the term “acidic polysaccharide” refers to polymeric carbohydrates including carboxylic acid groups. The polymeric carbohydrate can be naturally occurring, or can be synthetic or semi-synthetic. Examples of acidic polysaccharides are hyaluronan and carboxymethyl cellulose. An oxidized hyaluronan, that is, hyaluronan that has been treated with an oxidizing agent, such as sodium periodate, that cleaves vicinal diol moieties and provides aldehyde groups, is an acidic polysaccharide within the meaning herein, and is also an oxidized polysaccharide within the meaning herein.

As used herein, the term “acrylated chitosan” refers to a sample formed of chitosan molecules to which acrylate containing moieties have been bonded. The term “acrylated chitosan” thus includes an enormous number of possible chemical structures, but they all share the unifying feature that chemical bonds have been formed between the components of the chitosan molecules and at least one carbon atom in each of the acrylate molecules that are bonded to the chitosan.

As used herein, the terms “adhere” or “adherence” refers to the creation of a physical bond between the biocompatible adherent sheet and tissue such that a moderate motion or force does not cause separation of the biocompatible adherent sheet from the tissue on which it is disposed. Thus, a biocompatible adherent sheet serves to glue together living tissue, at least temporarily, such as for the amount of time it takes healing to occur. However, sealing may take place for a more prolonged period. The physical bond that is created between the biocompatible adherent sheet and the tissue that is being sealed may have one or several bases including electrostatic bonding and covalent bonding, but any mechanism by which the adherence takes place falls within the definition herein.

As used herein, the terms “adhesive” and “adhesivity” similarly refer to the existence of a physical bond between two materials such as a biocompatible adherent sheet and the tissue to which the biocompatible adherent sheet is applied. An adhesive is a material which adheres to tissue or other material and which may be used to constrain the separation of two tissue masses. Adhesivity is the property or degree to which a material adheres to a tissue or other material. In the biocompatible adherent sheets described herein, the modified chitosan acts as an adhesive material to bind the biocompatible adherent sheet to the tissue.

As used herein, the term “alkylated chitosan” refers a material formed of chitosan molecules to which carbon-containing molecules have been bonded. The term “alkylated chitosan” thus includes a large number of possible chemical structures, but they all share the unifying feature that chemical bonds have been formed between the components of the chitosan molecules and at least one carbon atom in each of the molecules that are bonded to the chitosan. For example, methylation of chitosan, in which bonds are formed between methyl radicals or groups and atoms within the chitosan molecule, such as nitrogen, oxygen or carbon atoms, provides an alkylated chitosan within the definition used herein. Other carbon-containing groups may likewise be chemically bonded to chitosan molecules to produce an alkylated chitosan. Specific examples include poly(oxyalkylene)chitosan (e.g., poly(oxyethylene) or polyethyleneglycol) chains covalently bonded to the chitosan backbone, as well as acrylated chitosans, formed by alkylation of chitosan with acrylates.

As used herein, the term “acrylated chitosan” refers a material in which acrylate moieties are bonded to the chitosan molecule, for example, via Michael addition of the chitosan amino groups to the acrylate β-carbons.

As used herein, the term “anti-cancer agent” refers to an agent that either inhibits the growth of cancerous cells, or causes the death of cancerous cells. Anti-cancer agents include, e.g., nucleotide and nucleoside analogs, such as 2-chloro-deoxyadenosine, adjunct antineoplastic agents, alkylating agents, nitrogen mustards, nitrosoureas, antibiotics, antimetabolites, hormonal agonists/antagonists, androgens, antiandrogens, antiestrogens, estrogen & nitrogen mustard combinations, gonadotropin releasing hormone (GNRH) analogues, progestrins, immunomodulators, miscellaneous antineoplastics, photosensitizing agents, and skin & mucous membrane agents. See, Physician's Desk Reference, 55^{th} Edition, Medical Economics, Montvale, N.J., USA (2001).

As used herein, the term “antimicrobial” refers to a molecular entity that is effective as a therapeutic agent or as a protective agent against an infection by a microorganism, which could be a bacterium, a protozoan, a fungus, a virus, or another pathogenic living organism. An antimicrobial may be an antibiotic, effective against bacteria, including aminoglycoside antibiotics such as gentamicin or streptomycin, a cephalosporin such as cephalexin or ceftriaxone, a carbacephem such as loracarbef, a glycopeptide such as vancomycin, a macrolide such as erythromycin, a penicillin such as amoxicillin or ampicillin, a polypeptide such as bacitracin or polymyxin B, a quinolone such as ciprofloxacin, a tetracycline such as oxytetracycline, a sulfonamide, or any other medically approved agent for treatment of bacterial infections. Alternatively the antimicrobial may be an antifungal agent such as ketoconazole, miconazole or amphotericin B, or an antiviral agent such as acyclovir or AZT.

As used herein, the term “aqueous” refers to a liquid mixture containing water, among other components.

As used herein, the term “bioactive agent” refers to a substance used in an application that is therapeutic in nature, such as methods for treating disease in a patient.

As used herein, the term “biocompatible” refers to the material, substance, compound, molecule, polymer, or system, which does not cause severe toxicity, severe adverse biological reaction, or lethality in an animal when administered at reasonable doses and rates.

As used herein, the term “carbodiimide” refers to a class of organic substances comprising an R—N═C═N—R′ moiety. The R and R′ groups may be any organic radicals. For example, when R and R′ are cyclohexyl groups, the carbodiimide is 1,3-dicyclohexylcarbodiimide, a dehydrating reagent well known in the art. A water-soluble carbodiimide is a carbodiimide that has sufficient solubility in water to form a homogeneous solution at concentrations suitable to carry out the gelation reaction as described herein. Typically, a water-soluble carbodiimide contains an ionic group, such as an ammonium salt, to confer water-solubility upon the molecule. The water-soluble carbodiimide EDCI is 1-ethyl-3-N,N-dimethylaminopropylcarbodiimide.

As used herein, the term “carbohydrate” refers to sugars or sugar derivatives with beta (β) or alpha (α) anomeric stereochemistry; moreover, the sugars can have (R) or (S) relative configurations, can exist as the (+) or (−) isomer, and can exist in the D or L configuration. The terms “anomer” and “anomeric” refer to the stereochemical configuration at the acetal, hemiacetal, or ketal carbon atom, as is well known in the art.

As used herein, the term “carboxyl activating reagent” refers to a molecular species that interacts with a carboxyl group in such a way as to render the carbonyl of the carboxyl group more susceptible to nucleophilic attack, as by an amine to yield an amide. This activation may take place by formation of a complex or by formation of a covalent intermediate. A specific example of a carboxyl activating reagent is an N-hydroxy compound that can form an N-hydroxy ester of the carboxylic acid group, increasing the reactivity of the carbonyl moiety to nucleophilic addition of a molecular species such as an amine.

As used herein, the term “carboxymethylcellulose” refers to a derivative of cellulose (a β-1,4 linked polymer of glucose) wherein hydroxyl groups are substituted with carboxymethyl (—CH₂CO₂H) moieties. It is understood that the term carboxymethylcellulose includes salts of carboxymethylcellulose, such as the sodium salt. Carboxymethylcellulose, as is well-known in the art, may have varying degrees of substitution, a “degree of substitution” referring to the number of derivatizing groups, herein carboxymethyl, per each monomer unit on the average. A particularly preferred carboxymethylcellulose has a degree of substitution of about 0.7 and a molecular weight of about 80 kD.

As used herein, the term “chitosan” refers to a polysaccharide polymer, either obtained from a natural source such as chitin, or synthetically prepared. Chemically, chitosan is predominantly a polymer of β-1,4-linked 2-amino-2-deoxyglucose monomers. When prepared from a natural source, the usual natural source is chitin, a major constituent of the shells of crabs, shrimp and other arthropods. After isolation of chitin from its natural source, chitin is treated in a manner as to cause hydrolysis of the acetamido group without cleavage of the sugar-sugar bonds, typically through alkaline hydrolysis to provide chitosan. Chitosan is not a single molecular entity, but includes polymeric chains of various lengths.

As used herein, the term “conformable” refers to the capability of a material to macroscopically adapt to the overall shape of one or more surfaces of a tissue or body part. The tissue or body parts may include, for example planar, contoured surfaces (e.g., convex and/or concave), highly contoured surface such as spherical surfaces having a large radius of curvature, and irregular surfaces having complex contour such as surfaces having both convex and concave surface features.

As used herein, the term “degree of polymerization” of a polymeric species refers to the number of monomeric units in a given polymer molecule, or the average of such numbers for a set of polymer molecules.

As used herein, the term “degree of substitution” of a polymeric species refers to the ratio of the average number of substituent groups, for example an alkyl substituent, per monomeric unit of the polymer as defined.

As used herein, the term “dehydrating reagent” refers to a molecular species that takes up the elements of water from a reaction, serving to drive a coupling reaction by thermodynamic factors. A dehydrating reagent is a compound that undergoes reaction of covalent bonds upon taking up the elements of water, as opposed to merely absorbing water into physical particles or the like. Preferably a dehydrating reagent is an organic compound. A specific example of a dehydrating reagent is a carbodiimide that takes up the elements of water and undergoes changes in covalent bonds to ultimately yield a urea derivative.

As used herein, the term “drug” refers to a chemical capable of administration to an organism which modifies or alters the organism's physiology. More preferably, as used herein, the term “drug” refers to any substance intended for use in the treatment or prevention of disease, particularly for humans. Drug includes synthetic and naturally occurring toxins and bioaffecting substances as well as recognized pharmaceuticals, such as those listed in The Merck Index, 14^{th} Ed., Merck Research Laboratories, Whitehouse Station, N.J., 2006, The Physicians Desk Reference, 62^{nd} edition, 2008, pages 101-201, Thomson Healthcare Inc., Montvale, N.J.; Goodman and Gilman's The Pharmacological Basis of Therapeutics, 8^{th} Edition (1990), pages 84-1614 and 1655-1715; and The United States Pharmacopeia, The National Formulary, USP XXII NF XVII (1990), the compounds of these references being herein incorporated by reference.

Specifically, the drug can include, but is not limited to, one or more polynucleotides, polypeptides, oligonucleotides, gene therapy agents, nucleotide analogs, nucleoside analogs, polynucleic acid decoys, therapeutic antibodies, anti-inflammatory agents, blood modifiers, anti-platelet agents, anti-coagulation agents, immune suppressive agents, anti-neoplastic agents, anti-cancer agents, anti-cell proliferation agents, and nitric oxide releasing agents.

The polynucleotide can include deoxyribonucleic acid (DNA), ribonucleic acid (RNA), double stranded DNA, double stranded RNA, duplex DNA/RNA, antisense polynucleotides, functional RNA or a combination thereof. In one embodiment, the polynucleotide can be RNA. In another embodiment, the polynucleotide can be DNA. In another embodiment, the polynucleotide can be an antisense polynucleotide.

The polynucleotide can be a single-stranded polynucleotide or a double-stranded polynucleotide. The polynucleotide can have any suitable length. Specifically, the polynucleotide can be about 2 to about 5,000 nucleotides in length, inclusive; about 2 to about 1000 nucleotides in length, inclusive; about 2 to about 100 nucleotides in length, inclusive; or about 2 to about 10 nucleotides in length, inclusive.

An antisense polynucleotide is typically a polynucleotide that is complimentary to an mRNA, which encodes a target protein. For example, the mRNA can encode a cancer promoting protein i.e., the product of an oncogene. The antisense polynucleotide is complimentary to the single stranded mRNA and will form a duplex and thereby inhibit expression of the target gene, i.e., will inhibit expression of the oncogene. The antisense polynucleotides of the invention can form a duplex with the mRNA encoding a target protein and will disallow expression of the target protein.

As used herein, the term “effective amount” refers to an amount of bioactive agent or any combination of bioactive agents useful to treat or prevent the underlying disorder or disease, or to treat the symptoms associated with the underlying disorder or disease in a host. Synergy occurs when the effect of several bioactive agents when administered in combination is greater than the additive effect of the bioactive agents, when administered alone as a single agent. In general, a synergistic effect is most clearly demonstrated at suboptimal concentrations of the bioactive agent. Synergy can be in terms of lower cytotoxicity, increased activity, or some other beneficial effect of the combination compared with the individual components.

As used herein, the term “immersing” refers to dipping, plunging, or sinking into a liquid.

As used herein, the term “flexible” refers to a biocompatible adherent sheet that is compliant and readily conforms to the general shape and contours of the tissues and parts of, for example, the human body.

As used herein, the term “gene therapy agent” refers to an agent that causes expression of a gene product in a target cell through introduction of a gene into the target cell followed by expression. An example of such a gene therapy agent would be a genetic construct that causes expression of a protein, such as insulin, when introduced into a cell. Alternatively, a gene therapy agent can decrease expression of a gene in a target cell. An example of such a gene therapy agent would be the introduction of a polynucleic acid segment into a cell that would integrate into a target gene and disrupt expression of the gene. Examples of such agents include viruses and polynucleotides that are able to disrupt a gene through homologous recombination. Methods of introducing and disrupting genes with cells are well known to those of skill in the art.

Nucleotide and nucleoside analogues are well known on the art. Examples of such nucleoside analogs include, but are not limited to, CYTOVENE (Roche Laboratories), EPIVIR (Glaxo Wellcome), GEMZAR (Lilly), HIVID (Roche Laboratories), REBETRON (Schering), VIDEX (Bristol-Myers Squibb), ZERIT (Bristol-Myers Squibb), and ZOVIRAX (Glaxo Wellcome). See, Physician's Desk Reference, 2007 Edition.

As used herein, the term “hemostasis” refers to those processes which comprise the defense mechanisms of the body against loss of circulating blood caused by vascular injury.

As used herein, the term “metabolite” refers to any compound produced in vivo or in vitro from a drug, or its prodrugs.

As used herein, the term “molecular weight” refers to a weight-average molecular weight, as is well known in the art.

As used herein, the term “N-hydroxy compound” refers to an organic compound comprising a chemical bond between a hydroxyl group and a nitrogen atom. Specific N-hydroxy compounds such as N-hydroxysuccinimide and N-hydroxybenzotriazole (1-hydroxy benzotriazole) are well known in the art as reagents that form esters with carboxylic acid groups and serve to activate the carboxylic acid group in reactions with nucleophiles.

As used herein, the term “oxidized polysaccharide” refers to a polymeric carbohydrate, acidic or non-acidic, that has undergone treatment with an oxidizing reagent, such as sodium periodate, that cleaves vicinal diol moieties of the carbohydrate to yield aldehyde groups. An oxidized hyaluronan, that is, hyaluronan that has been treated with an oxidizing agent, such as sodium periodate, that cleaves vicinal diol moieties and provides aldehyde groups, is an example of an acidic polysaccharide within the meaning herein. An oxidized dextran, that is, dextran that has been treated with an oxidizing agent, such as sodium periodate, that cleaves vicinal diol moieties and provides aldehyde groups, is another example of an oxidized polysaccharide within the meaning herein. Another example of an oxidized polysaccharide is an oxidized starch, that is, a starch that has been treated with an oxidizing agent, such as sodium periodate, that provides aldehyde groups. It is believed that the aldehyde groups of oxidized polysaccharides interact with the amino groups of an alkylated chitosan in such a way as to markedly increase the viscosity of the mixture and cause gelation. While not wishing to be bound by theory, it is believed that this intermolecular interaction takes place through the formation of imines, or Schiff bases, between the amino groups and the aldehyde groups.

As used herein, the terms “pharmaceutically acceptable salts” or “acceptable salts” refer to derivatives wherein the parent compound is modified by making acid or base salts thereof. Examples of acceptable salts include, but are not limited to, mineral or organic acid salts of basic residues such as amines; alkali or organic salts of acidic residues such as carboxylic acids; and the like. The acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like. Specifically, the acceptable salts can include those salts that naturally occur in vivo in a mammal.

As used herein, the terms “peptide” and a “protein” refer to polypeptides, linear polymers of amino acids, the difference between the terms “peptide” and “protein” largely being in the length of the polymer. In one embodiment, the polypeptide can be an antibody. Examples of such antibodies include single-chain antibodies, chimeric antibodies, monoclonal antibodies, polyclonal antibodies, antibody fragments, Fab fragments, IgA, IgG, IgM, IgD, IgE and humanized antibodies. In one embodiment, the antibody can bind to a cell adhesion molecule, such as a cadherin, integrin or selectin. In another embodiment, the antibody can bind to an extracellular matrix molecule, such as collagen, elastin, fibronectin or laminin. In still another embodiment, the antibody can bind to a receptor, such as an adrenergic receptor, B-cell receptor, complement receptor, cholinergic receptor, estrogen receptor, insulin receptor, low-density lipoprotein receptor, growth factor receptor or T-cell receptor. Antibodies of the invention can also bind to platelet aggregation factors (e.g., fibrinogen), cell proliferation factors (e.g., growth factors and cytokines), and blood clotting factors (e.g., fibrinogen). In another embodiment, an antibody can be conjugated to an active agent, such as a toxin.

As used herein, the term “polymer” refers to a molecule of one or more repeating monomeric residue units covalently bonded together by one or more repeating chemical functional groups. The term includes all polymeric forms such as linear, branched, star, random, block, graft, and the like. It includes homopolymers formed from a single monomer, copolymer formed from two or more monomers, terpolymers formed from three or more polymers, and polymers formed from more than three monomers. Differing forms of a polymer may also have more than one repeating, covalently bonded functional group.

As used herein, the term “poly(oxyalkylene)chitosan” refers to a variety of alkylated chitosan as defined herein. A “poly(oxyalkylene)” group is a polymeric chain of atoms wherein one or more carbon atoms are bonded at either end to oxygen atoms. The carbon atoms of the alkylene group may themselves bear additional radicals. For example, if an ethylene group bears a single methyl group, the resulting poly(oxyalkylene) group is a poly(oxypropylene) group. If the ethylene groups are unsubstituted, the poly(oxyalkylene) group is a poly(oxyethylene) group. A poly(oxyethylene) group may be of a wide range of lengths, or degrees of polymerization, but is of the general molecular formula of the structure [—CH₂—CH₂—O—CH₂—CH₂—O—]ₙ, where n may range from about 3 upwards to 10,000 or more. Commonly referred to as “polyethyleneglycol” or “PEG” derivatives, these polymeric chains are of a hydrophilic, or water-soluble, nature. Thus, a poly(oxyalkylene)chitosan is a chitosan derivative to which poly(oxyalkylene) groups are covalently attached. A terminal carbon atom of the poly(oxyalkylene) group forms a covalent bond with an atom of the chitosan chain, likely a nitrogen atom, although bonds to oxygen or even carbon atoms of the chitosan chain may exist. Poly(oxyethylene)chitosan is often referred to as “polyethyleneglycol-grafted chitosan” or “PEG-chitosan” or “PEG-g-chitosan” or “PEG-grafted-chitosan.”

As used herein, the term “prodrug” refers to any pharmaceutically acceptable form of compound of a drug which, upon administration to a patient, provides a drug. Pharmaceutically acceptable prodrugs refer to a compound that is metabolized, for example hydrolyzed or oxidized, in the host to form a drug. Typical examples of prodrugs include compounds that have biologically labile protecting groups on a functional moiety of the active compound. Prodrugs include compounds that can be oxidized, reduced, aminated, deaminated, hydroxylated, dehydroxylated, hydrolyzed, dehydrolyzed, alkylated, dialkylated, acylated, deacylated, phosphorylated, dephosphorylated to produce the active compound.

As used herein, the terms “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

As used herein, the term “saccharide” refers to a carbohydrate. The term “carbohydrate” includes the class of compounds commonly known as sugars, in addition to compounds that are chemically related to sugars. The term thus includes simple “monosaccharide” sugars, “disaccharide” sugars as well as polymeric “polysaccharides.” The term encompasses a group of compounds including sugars, starches, gums, cellulose and hemicellulose. The term further encompasses sugar derivatives such as amino-sugars, for example, 2-amino-2-deoxyglucose, as well as their oligomers and polymers; sulfated sugars; and sugars with hydroxyl, amino, carboxyl and other groups.

As used herein, the term “substantially uniform thickness” refers to a carrier sheet that has a thickness that is within 25% across the entire carrier sheet.

As used herein, the term “spraying” refers to scattering or throwing a liquid in a form of a spray.

As used herein, the term “to seal” or “sealing” refers to the act wherein two physically noncontiguous tissues or portions thereof are joined together, or where a hole, tear, cut, perforation or other discontinuity is repaired so as to close the hole, tear, cut or perforation. Sealing implies at least some degree of adhesion of the material, for example, a biocompatible adherent sheet to the tissue to which the biocompatible adherent sheet is applied, such that the sealed tissue is secured against at least a moderate displacing force. The discontinuity in the tissue that is being sealed may be an incision made as part of a surgical procedure, or it may be a wound.

As used herein, the term “tissue” refers to the material forming the solid or semi-solid structures that make up any of the organs or components of a living organism, preferably human. Thus, liquids such as blood are not “tissue” according to the definition used herein, but the term “tissue” encompasses membranes, skin, muscles, bones, cartilage, nerves and nerve sheathes, meninges, connective tissue, blood vessels, the sclera or iris of the eye, the solid materials constituting internal organs such as liver, stomach, pancreas, intestine, kidney, thymus, uterus, testes, bladder, lung, heart and any other internal structures that are solid or semi-solid in texture.

As used herein, the term “vascular system” refers to the system of vessels and tissues that carry or circulate fluids such as blood or lymph throughout a living mammalian body. The term “vascular” refers to the vascular system. A “vascular site” is a discrete location within the vascular system or a relatively small section of a vascular vessel or duct.

As used herein, “μg” denotes microgram, “mg” denotes milligram, “g” denotes gram, “μL” denotes microliter, “mL” denotes milliliter, “L” denotes liter, “nM” denotes nanomolar, “μM” denotes micromolar, “mM” denotes millimolar, “M” denotes molar, and “nm” denotes nanometer.

Concentrations, amounts, etc., of various components are often presented in a range format throughout this disclosure. The description in range format is merely for convenience and brevity and should not be construed as an inflexible limitation on the scope of the claimed invention. Accordingly, the description of a range should be considered to have specifically disclosed all the possible subranges as well as individual numerical values within that range. For example, description of a range such as 1% to 8% should be considered to have specifically disclosed subranges such as 1% to 7%, 2% to 8%, 2% to 6%, 3% to 6%, 4% to 8%, 3% to 8% etc., as well as individual numbers within that range, such as, 2%, 5%, 7% etc. This construction applies regardless of the breadth of the range and in all contexts throughout this disclosure.

In the claims provided herein, the steps specified to be taken in a claimed method or process may be carried out in any order without departing from the principles of the invention, except when a temporal or operational sequence is explicitly defined by claim language. Recitation in a claim to the effect that first a step is performed then several other steps are performed shall be taken to mean that the first step is performed before any of the other steps, but the other steps may be performed in any sequence unless a sequence is further specified within the other steps. For example, claim elements that recite “first A, then B, C, and D, and lastly E” shall be construed to mean step A must be first, step E must be last, but steps B, C, and D may be carried out in any sequence between steps A and E and the process of that sequence will still fall within the four corners of the claim.

Furthermore, in the claims provided herein, specified steps may be carried out concurrently unless explicit claim language requires that they be carried out separately or as parts of different processing operations. For example, a claimed step of doing X and a claimed step of doing Y may be conducted simultaneously within a single operation, and the resulting process will be covered by the claim. Thus, a step of doing X, a step of doing Y, and a step of doing Z may be conducted simultaneously within a single process step, or in two separate process steps, or in three separate process steps, and that process will still fall within the four corners of a claim that recites those three steps.

Similarly, except as explicitly required by claim language, a single substance or component may meet more than a single functional requirement, provided that the single substance fulfills the more than one functional requirement as specified by claim language.

### Carrier Sheet

The biocompatible adherent sheet may include, for example, a carrier sheet to serve as a support for the modified chitosan. The carrier sheet may include any biocompatible polymer. The biocompatible polymer made be a naturally occurring polymer, a synthetic polymer, or a combination thereof. Preferably, the biocompatible polymer is biodegradable.

Suitable naturally-occurring biocompatible polymers may include, for example, fibrous or globular proteins, complex carbohydrates, glycosaminoglycans, or combinations thereof. The naturally-occurring biocompatible polymers may include, for example, collagens of all types, elastin, hyaluronic acid, alginic acid, desmin, versican, matricelluar proteins such as osteonectin, osteopontin, thrombospondin 1 and 2, fibrin, fibronectin, vitronectin, albumin, or combinations thereof. The naturally-occurring biocompatible polymers may be used as the main scaffold for the carrier sheet or as an additive to improve the biocompatibility of a synthetic polymer acting as a scaffold for the carrier sheet.

Suitable synthetic biocompatible polymers may include, for example, 2-hydroxyethyl methacrylate, silicone rubber, poly(ε-caprolactone) dimethylacrylate, polysulfone, (poly)methyl methacrylate, soluble Teflon-AF, polyethylene terephthalate, nylon, polyvinyl alcohol, polyurethane, or combinations thereof.

Preferably, the biocompatible polymer includes hyaluronic acid, polyhydroxy acid, lactic acid, glycolic acid, hydroxybutanoic acid, cellulose, gelatin, collagen, or a combination thereof. Most preferably, the biocompatible polymer is collagen.

The collagen may be from any class, for example, Class I, II, III, IV, etc. The collagen may be from any source, for example, a mammalian, transgenic, recombinant source, or combination thereof. If the collagen is from a non-human mammal, in order to prevent virus transmission due to contamination with animal viruses that are pathogenic to humans by virtue of the carrier sheet, appropriate selection of source material and inactivation of potentially pathogenic agents by the manufacturing process is important as precautionary measures.

The collagen may be uncrosslinked (0% linkages), partially crosslinked (greater than 0% and less than 100% linkages), or fully crosslinked (100% linkages). Chemical crosslinking may be introduced in an amount sufficient to make the collagen substantially non-resorbable or biodegradable and non-bioactive. One skilled in the art appreciates that the non-resorption or permanency of collagen increases with the amount of crosslinked bonds. For example, in a highly crosslinked collagen having 85% crosslinked bonds, the collagen may remain substantially intact inside of a recipient for months, decades, or a lifetime. Furthermore, the percentage of crosslinked bonds may ensure that the substantial majority of the collagen does not degrade, deform, or otherwise lose strength over the life of the biocompatible adherent sheet. In contrast, a lesser crosslinked collagen having about 10% linkage, may be for temporary use and designed to retain the majority of its structural integrity for only a few weeks or months. This may be useful in situations where the repair is minor and may be replaced with regenerated tissue in a short time period.

The collagen may be uncrosslinked or partially or fully crosslinked using, for example, chemical crosslinking, ultraviolet radiation, dehydrothermal crosslinking, or combinations of these treatments. Chemical crosslinking may be performed using a chemical crosslinking agent, including, for example, carbodiimide, glutaraldehyde, formaldehyde, diisocyanates, and mixtures thereof. The crosslinking is carried out for a time and under conditions sufficient to provide a non-immunogenic collagen. In embodiments where a greater degree of crosslinking is desired, the duration of the crosslinking treatment may increase or a successive series of crosslinking treatments may be used.

The carrier sheet may also include a mixture of biocompatible polymers that biodegrade at different rates. In one embodiment, the carrier sheet may include a combination of slow-biodegrading collagen and a slow-biodegrading polymer. In another embodiment, the carrier sheet may include a combination of slow-biodegrading collagen and a fast-biodegrading polymer. In yet another embodiment, the carrier sheet may include a combination of fast-biodegrading collagen and a slow-biodegrading polymer. In still yet another embodiment, the carrier sheet may include a combination of fast-biodegrading collagen and a fast-biodegrading polymer.

In one embodiment, the other biodegrading polymer elicits a positive tissue response. In another embodiment, the other biodegrading polymer does not elicit a positive tissue response.

In one embodiment, a fast-biodegrading polymer that elicits a positive tissue response may be used with a slow-biodegrading collagen. This combination may allow newly generated tissues to develop into the slow-biodegrading collagen.

In one embodiment, the carrier sheet may also include a modified chitosan as described herein.

The carrier sheet should be flexible, conformable, and of substantially uniform thickness. The carrier sheet should be flexible and readily assume the general shape and contours of the tissues and parts of, for example, the human body. The carrier sheet may also have elastic characteristics allowing it to stretch in one or two directions. The carrier sheet should also be conformable and capable of adapting to the overall shape of the tissues and parts of, for example, the human body, via intimate contact without creating voids or kinks. Further, the carrier sheet should have a substantially uniform thickness across its longitudinal (y) and transverse (x) directions. By “substantially uniform thickness” is meant that the thickness of the carrier sheet that has a thickness that is within 25% across the entire carrier sheet.

The carrier sheet may be of any thickness such that the carrier sheet can readily assume the general shape and contours of the tissues and parts of, for example, the human body. One of skill in the art would easily recognize that the thickness of the carrier sheet would depend upon the particular application. Typically, the carrier sheet has a thickness of about 0.001 mm to about 100 mm, preferably from about 0.1 mm to about 50 mm.

The carrier sheet may have any density. One of skill in the art would easily recognize that the density of the carrier sheet would depend upon the particular application. Typically, the carrier sheet has a density of about 1 mg/cm³ to about 100 mg/cm³.

### Modified Chitosan

The modified chitosan may be used to adhere the carrier sheet to the one or more moist body tissue surfaces. The modified chitosan may be an alkylated chitosan, the reaction product of an alkylated chitosan and an acidic polysaccharide, the reaction product of an alkylated chitosan and an oxidized polysaccharide, the reaction product of an alkylated chitosan, an acidic polysaccharide, and an oxidized polysaccharide, or the reaction product of an alkylated chitosan, an acidic polysaccharide, a carboxyl activating reagent, and a dehydrating reagent, wherein the alkylated chitosan includes an acrylated chitosan or a poly(oxyalkylene)chitosan.

Specific examples of alkylated chitosan include poly(oxyalkylene)chitosan, wherein poly(oxyethylene), or polyethyleneglycol, chains are covalently bonded to the chitosan backbone, as well as acrylated chitosans, formed by alkylation of chitosan with acrylates. Preferably, the modified chitosan includes an alkylated chitosan, the reaction product of an alkylated chitosan and an acidic polysaccharide, the reaction product of an alkylated chitosan and an oxidized polysaccharide, the reaction product of an alkylated chitosan, an acidic polysaccharide, and an oxidized polysaccharide, or the reaction product of an alkylated chitosan, an acidic polysaccharide, a carboxyl activating reagent, and a dehydrating reagent, wherein the alkylated chitosan includes an acrylated chitosan or a poly(oxyalkylene)chitosan. More preferably, the modified chitosan includes an acrylated chitosan or the reaction product of a poly(oxyethylene)chitosan and hyaluronic acid.

The acrylated chitosan is an alkylated chitosan wherein acrylates have been allowed to react with, and form chemical bonds to, the chitosan molecule. An acrylate is a molecule containing an α,β-unsaturated carbonyl group; thus, acrylic acid is prop-2-enoic acid. An acrylated chitosan is a chitosan wherein a reaction with acrylates has taken place. The acrylate may bond to the chitosan through a Michael addition of the chitosan nitrogen atoms with the acrylate.

A preferred degree of substitution of the chitosan backbone with acrylate groups is about 0.25 to about 0.45. The number of monomeric units that make up a acrylated chitosan may vary widely. Any sample that contains more than a single molecule of a chitosan derivative should almost inevitably contain a distribution of molecules of different molecular weights. A preferred acrylated chitosan has a molecular weight of about 200 kD to about 600 kD. Preferably, the acrylated chitosan includes an N-acrylated chitosan.

Another suitable modified chitosan may be the reaction product of a poly(oxyalkylene)chitosan and an acidic polysaccharide. The poly(oxyalkylene)chitosan is a polymer formed of 2-amino-2-deoxyglucose monomeric units. Each monomeric unit includes a single free amino group and two free hydroxyl groups. One amino group is alkylated on the nitrogen atom with a poly(oxyethylene) chain. Preferably, the poly(oxyalkylene)chitosan is poly(oxyethylene)chitosan.

Another suitable modified chitosan may be the reaction product of an alkylated chitosan and an oxidized polysaccharide.

Another suitable modified chitosan may be the reaction product of an alkylated chitosan, an acidic polysaccharide, and an oxidized polysaccharide.

Another suitable modified chitosan may be the reaction product of an alkylated chitosan, an acidic polysaccharide, a carboxyl activating reagent, and a dehydrating reagent.

In one embodiment, the chitosan has a degree of substitution of 0.5, because two of the four amino groups in the tetrameric unit shown bear the substituent. However, a poly(oxyethylene)chitosan may also have a degree of amino group substitution ranging down to about 0.1 (wherein only one in about every ten monomeric units is alkylated). Furthermore, a poly(oxyethylene)chitosan may also bear the poly(oxyethylene) derivative on one of the two free hydroxyl groups in a given monomeric unit, or may includes a mixture of N- and O-alkylated chitosan monomeric units, or be di-alkylated or tri-alkylated on a single monomer unit. Thus, a fully alkylated chitosan monomeric unit has a degree of substitution of 3.0, and poly(oxyethylene)chitosan may have a degree of substitution ranging up to 3.0. A preferred degree of substitution for a poly(oxyethylene)chitosan is about 0.35 to about 0.95. A particularly preferred degree of substitution is about 0.5. It should be understood that other poly(oxyalkylene) groups may be used. For example, a poly(oxypropylene)chitosan may be used. A poly(oxypropylene) group is the structure that would be obtained if the poly(oxyethylene) group bore a methyl group on every ethylene unit (—O—CH₂CH(CH₃)—O), or alternatively, every ethylene unit were a 3-carbon linear propylene group (—O—CH₂CH₂CH₂—O—).

The number of monomeric units that make up a modified chitosan may vary widely without departing from the principles of the invention. Any sample that contains more than a single molecule of a chitosan derivative should almost inevitably contain a distribution of molecules of different molecular weights. Preferably, the poly(oxyalkylene)chitosan includes a poly(oxyethylene)chitosan. A preferred poly(oxyethylene)chitosan has a molecular weight of about 200 kD to about 600 kD.

Suitable acidic polysaccharides may include, for example, naturally-occurring acidic polysaccharides, synthetic polysaccharides, or combinations thereof.

Suitable naturally-occurring acidic polysaccharides may include, for example, sualginic acid, alginic acid, pectic acid, hyaluronic acid, xanthane, fucoidan, sulfated fucogalactan, sulfated fucoglucuronomannan, glucuronoxylofucane, sargassan, glucuronomannogalactan, xylofucoglucuronan, ascorfilan, glucuronogalactofucane, sulfated glucuronofucane, sulfated galactan, carrageenan, funoran, agaropectin, rhaman sulfate, polygalacturonic acids, and the like, or combinations thereof.

Suitable synthetic acidic polysaccharide may include, for example, carboxymethyl cellulose, sulfates of cellulose, starch, mannan, xylan, alginic acid, pectin, pectic acid, hyaluronic acid, fructan, arabinan, chitin, pullulan, xyloglucan, dextran, synthetic sulfated polysaccharides such as ribofuranan sulfate, xylofuranan sulfate, lentinan sulfate, curdlan sulfate, mannopyranan sulfate, sulfated starch and sulfated pectin, and synthetic sulfated alkyl polysaccharides such as a ribofuranan sulfate, and the like, or combinations thereof. Preferably, the acidic polysaccharide may be a hyaluronan or a carboxymethylcellulose.

Hyaluronic acid bears an ionizable carboxylic acid group on every other monosaccharide residue. Hyaluronic acid can be in the form of a hyaluronate, that is, with at least most of the carboxylic acid groups being in the ionized or salt form. Sodium hyaluronate is a specific example. A hyaluronan or a hyaluronic acid is a polybasic carboxylic acid, and the number of ionizable carboxylate groups per hyaluronan molecule is dependent on the degree of polymerization of the hyaluronan. The degree of substitution of carboxylic acid groups on the polymer backbone, assuming a monomeric unit including the disaccharide formed of one glucaronic acid monosaccharide and one 2-acetamido-2-deoxyglucose monosaccharide, is 1.0. Every monomeric unit (disaccharide unit) bears a single ionizable carboxylic acid group. A hyaluronan may be of any of a wide range of degrees of polymerization (molecular weights), but a preferred hyaluronan has a molecular weight of about 2,000 kD to about 3,000 kD.

Carboxymethylcellulose, as is well-known in the art, may have varying degrees of substitution, a “degree of substitution” referring to the number of derivatizing groups, herein carboxymethyl, per each monomer unit on the average. A particularly preferred carboxymethylcellulose has a degree of substitution of about 0.7 and a molecular weight of about 80 kilodaltons (kD).

The modified chitosan substantially evenly disposed on one or both surfaces of the carrier sheet. By “substantially evenly disposed” is meant that the thickness of the modified chitosan on the carrier sheet is within 25% across the entire carrier sheet.

In one embodiment, the modified chitosan is distributed on one surface of the carrier sheet. In another embodiment, the modified chitosan is distributed on both surfaces of the carrier sheet.

The modified chitosan may be present in any amount necessary to provide adhesion to body surfaces. Typically, the modified chitosan is present in an amount of about 0.1 mg/cm² to about 100 mg/cm².

### Bioactive Agent

A bioactive agent may be included in the biocompatible adherent sheet. The bioactive agent may be included into the carrier sheet, the modified chitosan, or both the carrier sheet and the modified chitosan.

The bioactive agent may include, for example, a hormone, an immunomodulator, an immunosuppressant, an antibiotic, a cytostatic, a diuretic, a gastrointestinal agent, a cardiovascular agent, a neuropharmaceutical, a blood coagulation inducing agent, or a combination thereof.

The bioactive agent may also include, for example, any drug, metabolite, or prodrug thereof, organic compound, substance, nutrient or biologically beneficial agent including proteins, peptides (including polypeptides and oligopeptides), hormones, vaccines, oligonucleotides, genes, nucleic acids, steroids, antibiotics, antibodies, viruses, live cells, and other chemotherapeutic or non-therapeutic agents, or a combination thereof.

Suitable bioactive agents may also include, for example, a regenerative agent such as one or more human growth modulating factors such as interleukins, transformation growth factor-b, fibroblast growth factor or vascular endothelial growth factor; or the agent may be a gene therapy agent, a cogener of platelet derived growth factor, or a monoclonal antibody directed against growth factors; or the agent may be a drug, a cell regeneration factor, drug-producing cells, or regenerative cells.

Due to the abundance of cationic amino groups along the structure of chitosan, it is known that drugs with carboxyl groups may be conjugated thereto and sustained release can be achieved through the hydrolysis of the amide or ester bonds linking drugs to the chitosan molecule. As a polyelectrolyte, chitosan can also electrostatically conjugate sensitive bioactive agents (e.g., recombinant proteins, such as VEGF) while preserving their bioactivities and enhancing their stabilities. Such derivatives may be formed with the acrylated chitosan and should likewise serve to provide for sustained release and to preserve the bioactivity and to enhance the stability of the conjugated agent(s).

Suitable bioactive agents may further include, for example, progenitor cells of the same type as those from the vascular site, for example, an aneurysm, and progenitor cells that are histologically different from those of the vascular site such as embryogenic or adult stem cells, which can act to stabilize the vasculature and/or to accelerate the healing process. These cells may be incorporated into the carrier sheet, the modified chitosan, or both the carrier sheet and modified chitosan.

Additional bioactive agents may include, for example, one or more supplements, such as growth factors, polyclonal and monoclonal antibodies, and other compounds. Illustrative examples of such supplements include, for example, the following: fibrinolysis inhibitors, such as aprotonin, tranexamic acid and ε-amino-caproic acid; antibiotics, such as tetracycline and ciprofloxacin, amoxicillin, and metronidazole; anticoagulants, such as activated protein C, heparin, prostacyclins, prostaglandins (particularly (PGI₂), leukotrienes, antithrombin III, ADPase, and plasminogen activator; steroids, such as dexamethasone, inhibitors of prostacyclin, prostaglandins, leukotrienes and/or kinins to inhibit inflammation; cardiovascular drugs, such as calcium channel blockers, vasodilators and vasoconstrictors; chemo attractants; local anesthetics such as bupivacaine; and antiproliferative/antitumor drugs such as 5-fluorouracil, taxol and/or taxotere; antivirals, such as gangcyclovir, zidovudine, amantidine, vidarabine, ribaravin, trifluridine, acyclovir, dideoxyuridine and antibodies to viral components or gene products; cytokines, such as α- or β- or γ-Interferon, α- or β-tumor necrosis factor, and interleukins; colony stimulating factors; erythropoietin; antifungals, such as diflucan, ketaconizole and nystatin; antiparasitic gents, such as pentamidine; anti-inflammatory agents, such as α-1-anti-trypsin and α-1-antichymotrypsin; anesthetics, such as bupivacaine; analgesics; antiseptics; hormones; vitamins and other nutritional supplements; glycoproteins; fibronectin; peptides and proteins; carbohydrates (both simple and/or complex); proteoglycans; antiangiogenins; antigens; lipids or liposomes; oligonucleotides (sense and/or antisense DNA and/or RNA); and gene therapy reagents.

The amount of bioactive agent incorporated into the biocompatible adherent sheet depends upon the desired release profile, the concentration of bioactive agent required for a biological effect, and the length of time that the bioactive agent, has to be released for treatment, and should be within the discretion and wisdom of the patient's attending physician. There is no upper limit on the amount of bioactive agent incorporated into the biocompatible adherent sheet. The lower limit of bioactive agent incorporated into the biocompatible adherent sheet is dependent upon the activity of the bioactive agent, and the length of time needed for treatment. Specifically, in one embodiment, the biocompatible adherent sheet can be formulated to provide a one month release of bioactive agent. Alternatively, in another embodiment, the biocompatible adherent sheet can be formulated to provide a three month delivery of bioactive agent. The biocompatible adherent sheet should release the bioactive agent contained within the biocompatible adherent sheet at a controlled rate until the biocompatible adherent sheet is effectively depleted of bioactive agent.

A biocompatible adherent sheet may further comprise microspheres or nanospheres which preferably contain a therapeutic agent, the microspheres or nanospheres also controlling the release of the therapeutic agent into the surrounding tissues. As used herein, the terms “microsphere” or a “nanosphere” refer a particulate body of dimensions of the order of microns (micrometers) or nanometers respectively, wherein the particulate body may be hollow or solid. Microspheres and nanospheres may be formed of organic or inorganic materials. For example, a nanosphere may comprise a buckminsterfullerene (buckyball), which is organic. Alternatively a nanosphere may comprise microporous glass, which is inorganic. It is understood that the terms encompass solid lipid nanoparticles, wherein the nanosphere particles are formed from a solid lipid. Preferably the microsphere or the nanosphere contains a drug or other substance, the timing of the release of which it is advantageous to control.

In one embodiment, a biocompatible adherent sheet for application to one or more moist body tissue surfaces for obtaining hemostasis, tissue sealing, tissue gluing, and filling tissue voids is provided comprising: a carrier sheet comprising a biocompatible polymer and a modified chitosan, wherein the carrier sheet is flexible, conformable, and of substantially uniform thickness; and wherein the modified chitosan includes an acrylated chitosan or the reaction product of a poly(oxyalkylene)chitosan and an acidic polysaccharide.

In another embodiment, biocompatible adherent sheet for application to one or more moist body tissue surfaces for obtaining hemostasis, tissue sealing, tissue gluing, and filling tissue voids is provided comprising: a carrier sheet comprising a biocompatible polymer and a modified chitosan, wherein the carrier sheet is flexible, conformable, and of substantially uniform thickness; and a modified chitosan substantially evenly disposed on one surface of the carrier sheet, wherein the modified chitosan includes an acrylated chitosan or the reaction product of a poly(oxyalkylene)chitosan and an acidic polysaccharide.

In yet another embodiment, biocompatible adherent sheet for application to one or more moist body tissue surfaces for obtaining hemostasis, tissue sealing, tissue gluing, and filling tissue voids is provided comprising: a carrier sheet comprising a biocompatible polymer and a modified chitosan, wherein the carrier sheet is flexible, conformable, and of substantially uniform thickness; and a modified chitosan substantially evenly disposed on both surfaces of the carrier sheet, wherein the modified chitosan includes an acrylated chitosan or the reaction product of a poly(oxyalkylene)chitosan and an acidic polysaccharide.

In one embodiment, the biocompatible adherent sheet may also include, for example, a release layer in addition to the carrier sheet and modified chitosan. As used herein, a “release layer” refers to a layer containing one or more agents (“release agents”) which promote or facilitate removal of the biocompatible adherent sheet from a substrate in which it has been manufactured. Alternatively, such one or more release agents may be contained in the carrier sheet, the modified chitosan, or both the carrier sheet and the modified chitosan.

In one embodiment, the biocompatible adherent sheet may also include, for example, a release liner in addition to the carrier sheet and modified chitosan. As used herein, the term “release liner” refers to the non-stick backing that is applied to an uncoated side of the carrier or a side of the carrier sheet coated with the modified chitosan. The release liner aids in transport and storage of the biocompatible adherent sheet from the manufacturing site to the end user. The release liner provides protection for the biocompatible adherent sheet against exposure to materials, which might negatively affect the ability of the biocompatible adherent sheet to adhere to the shape and contours of the tissues and parts of, for example, the human body. The release liner may also provide protection against undesired, premature adhesion of the biocompatible adherent sheet to an unwanted material. Conventional release liners comprise a paper substrate coated with a release coating. The release coating is formulated to provide very little adhesion of the coated paper to any other substrate, particularly adhesive materials, so the release liner may be easily removed from the adhesive strip without disturbing the adhesive strip. Typically, release coatings include a silicone-containing polymeric material.

All patents, patent applications, publications, scientific articles, web sites, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Additionally, all claims in this application, and all priority applications, including but not limited to original claims, are hereby incorporated in their entirety into, and form a part of, the written description of the invention. Applicants reserve the right to physically incorporate into this specification any and all materials and information from any such patents, applications, publications, scientific articles, web sites, electronically available information, and other referenced materials or documents. Applicants reserve the right to physically incorporate into any part of this document, including any part of the written description, the claims referred to above including but not limited to any original claims.

### EXAMPLES

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements.

### Example 1

### Preparation of Biocompatible Adherent Sheet

2 milliliters (ml) of a 1 weight percent aqueous collagen suspension (bovine tendon derived collagen, Integra Life Sciences, Plainsburo, N.J., USA) in 0.01 normal (N) hydrochloric acid (HCl) was placed in a mold. The mold was frozen to about −20° C. to about −40° C. on a metal platform to afford a frozen collagen solution. 1 ml of about one weight percent aqueous acrylated chitosan solution was cast on the frozen collagen solution. The aqueous acrylated chitosan solution on the frozen collagen solution was frozen to about −20° C. to about −40° C. The frozen combination was lyophilized to afford a soft, pliable material that sticks to tissue. This soft, pliable material bends around curved surfaces. The biocompatible adherent sheet also conforms to the abdominal muscles of rabbits, rats and mice, chicken thighs, and to mice, rat, rabbit, dog, and pig livers. After application with gentle padding, the biocompatible adherent sheet adheres to body tissues within seconds after application. The biocompatible adherent sheet can withstand water pressures of 15 cm as defined in ASTM F2392-04.

### Example 2

### Preparation of Biocompatible Adherent Sheet

Example 1 was repeated using an aqueous PEG-chitosan-hyaluronan solution instead of the aqueous acrylated chitosan solution. The resulting biocompatible adherent sheet exhibited excellent adhesion to various body tissues.

### Example 3

### Preparation of Biocompatible Adherent Sheet

Example 1 is repeated using an aqueous acrylated chitosan and adipic acid solution instead of the aqueous acrylated chitosan solution.

### Example 4

### Preparation of Biocompatible Adherent Sheet

Example 1 is repeated using an aqueous acrylated chitosan and carboxymethylcellulose solution instead of the aqueous acrylated chitosan solution.

### Example 5

### Preparation of Biocompatible Adherent Sheet

Example 1 is repeated using an aqueous oxidized dextran/acrylated chitosan solution instead of the aqueous acrylated chitosan solution.

All patents and publications referenced or mentioned herein are indicative of the levels of skill of those skilled in the art to which the invention pertains, and each such referenced patent or publication is hereby incorporated by reference to the same extent as if it had been incorporated by reference in its entirety individually or set forth herein in its entirety. Applicants reserve the right to physically incorporate into this specification any and all materials and information from any such cited patents or publications.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. The methods and processes illustratively described herein suitably may be practiced in differing orders of steps, and that they are not necessarily restricted to the orders of steps indicated herein or in the claims.

What is claimed is:

1. A biocompatible adherent sheet for application to one or more moist body tissue surfaces for obtaining hemostasis, tissue sealing, tissue gluing, and filling tissue voids, comprising:
a carrier sheet comprising a biocompatible polymer, wherein the carrier sheet is flexible, conformable, and of substantially uniform thickness; and
a modified chitosan substantially evenly disposed on one or both surfaces of the carrier sheet,
wherein the modified chitosan comprises an alkylated chitosan, the reaction product of an alkylated chitosan and an acidic polysaccharide, the reaction product of an alkylated chitosan and an oxidized polysaccharide, the reaction product of an alkylated chitosan, an acidic polysaccharide, and an oxidized polysaccharide, or the reaction product of an alkylated chitosan, an acidic polysaccharide, a carboxyl activating reagent, and a dehydrating reagent,
wherein the alkylated chitosan comprises an acrylated chitosan or a poly(oxyalkylene)chitosan.

2. The biocompatible adherent sheet of claim 1, wherein the biocompatible polymer comprises hyaluronic acid, polyhydroxy acid, lactic acid, glycolic acid, hydroxybutanoic acid, cellulose, gelatin, collagen, or a combination thereof.

3. The biocompatible adherent sheet of claim 2, wherein the biocompatible polymer is collagen.

4. The biocompatible adherent sheet of claim 1, wherein the modified chitosan is distributed on one surface of the carrier sheet.

5. The biocompatible adherent sheet of claim 1, wherein the modified chitosan is distributed on both surfaces of the carrier sheet.

6. The biocompatible adherent sheet of claim 1, wherein the acrylated chitosan comprises an N-acrylated chitosan.

7. The biocompatible adherent sheet of claim 1, wherein the poly(oxyalkylene)chitosan comprises a poly(ethyleneglycol)chitosan.

8. The biocompatible adherent sheet of claim 1, wherein the acidic polysaccharide comprises a hyaluronan or a carboxymethylcellulose.

9. The biocompatible adherent sheet of claim 1, wherein the oxidized polysaccharide comprises an oxidized dextran, an oxidized starch, or an oxidized hyaluronan.

10. The biocompatible adherent sheet of claim 1, wherein the carboxyl activating reagent comprises an N-hydroxy compound.

11. The biocompatible adherent sheet of claim 1, wherein the dehydrating reagent comprises a carbodiimide.

12. The biocompatible adherent sheet of claim 1, wherein the moist body tissue surfaces are in a gastrointestinal system, parenchymal organs, a cardiovascular system, a thoracic system, a pulmonary system, an ear area, a nose area, a throat area, a dental area, a gynecological system, a urological system, a vascular system, a bone system, a neurological system, a lymphatic system, a dermal surface, a biliary system, or a combination thereof.

13. The biocompatible adherent sheet of claim 1, wherein the carrier sheet, the modified chitosan, or both the carrier sheet and the modified chitosan, further comprise a bioactive agent.

14. The biocompatible adherent sheet of claim 13, wherein the bioactive agent comprises a hormone, an immunomodulator, an immunosuppressant, an antibiotic, a cytostatic, a diuretic, a gastrointestinal agent, a cardiovascular agent, a neuropharmaceutical, a blood coagulation inducing agent, or a combination thereof.

15. A method of preparing a biocompatible adherent sheet for application to one or more moist body tissue surfaces for obtaining hemostasis, tissue sealing, tissue gluing, and filling tissue voids, the method comprising:
disposing a modified chitosan substantially evenly on one or both surfaces of a carrier sheet comprising a biocompatible polymer, wherein the carrier sheet is flexible, conformable, and of substantially uniform thickness, and
wherein the modified chitosan comprises an alkylated chitosan, the reaction product of an alkylated chitosan and an acidic polysaccharide, the reaction product of an alkylated chitosan and an oxidized polysaccharide, the reaction product of an alkylated chitosan, an acidic polysaccharide, and an oxidized polysaccharide, or the reaction product of an alkylated chitosan, an acidic polysaccharide, a carboxyl activating reagent, and a dehydrating reagent,
wherein the alkylated chitosan comprises an acrylated chitosan or a poly(oxyalkylene)chitosan.

16. The method of claim 15, wherein the biocompatible polymer comprises hyaluronic acid, polyhydroxy acid, lactic acid, glycolic acid, hydroxybutanoic acid, cellulose, gelatin, collagen, or a combination thereof.

17. The method of claim 16, wherein the biocompatible polymer is collagen.

18. The method of claim 15, wherein the acrylated chitosan comprises an N-acrylated chitosan.

19. The method of claim 15, wherein the poly(oxyalkylene)chitosan comprises a poly(ethyleneglycol)chitosan.

20. The method of claim 15, wherein the acidic polysaccharide comprises a hyaluronan or a carboxymethylcellulose.

21. The method of claim 15, wherein the oxidized polysaccharide comprises an oxidized dextran, an oxidized starch, or an oxidized hyaluronan.

22. The method of claim 15, wherein the carboxyl activating reagent comprises an N-hydroxy compound.

23. The method of claim 15, wherein the dehydrating reagent comprises a carbodiimide.

24. The method of claim 15, wherein the carrier sheet, the modified chitosan, or both the carrier sheet and the modified chitosan, further comprise a bioactive agent.

25. The method of claim 24, wherein the bioactive agent comprises a hormone, an immunomodulator, an immunosuppressant, an antibiotic, a cytostatic, a diuretic, a gastrointestinal agent, a cardiovascular agent, a neuropharmaceutical, a blood coagulation inducing agent, or a combination thereof.

26. A method of preparing a biocompatible adherent sheet for application to one or more moist body tissue surfaces, the method comprising:
casting an aqueous carrier solution comprising a biocompatible polymer;
freezing the aqueous carrier solution to provide a frozen aqueous carrier solution;
casting an aqueous modified chitosan solution on one or more surfaces of the frozen aqueous carrier solution;
freezing the aqueous modified chitosan solution to provide a frozen aqueous chitosan solution on one or more surfaces of the frozen aqueous carrier solution; and
lyophilizing the frozen aqueous modified chitosan solution on one or more surfaces of the frozen aqueous carrier solution,
wherein the modified chitosan comprises an alkylated chitosan, the reaction product of an alkylated chitosan and an acidic polysaccharide, the reaction product of an alkylated chitosan and an oxidized polysaccharide, the reaction product of an alkylated chitosan, an acidic polysaccharide, and an oxidized polysaccharide, or the reaction product of an alkylated chitosan, an acidic polysaccharide, a carboxyl activating reagent, and a dehydrating reagent,
wherein the alkylated chitosan comprises an acrylated chitosan or a poly(oxyalkylene)chitosan.

27. A method of preparing a biocompatible adherent sheet for application to one or more moist body tissue surfaces, the method comprising:
contacting a carrier sheet comprising a biocompatible polymer with an aqueous modified chitosan solution, wherein the carrier sheet is flexible, conformable, and of substantially uniform thickness, wherein the contacting comprises immersing, spraying, dipping, spin coating, bar coating, knife coating, extrusion coating, or direct coating;
freezing the carrier sheet contacted with the aqueous modified chitosan solution; and
lyophilizing the frozen carrier sheet contacted with the aqueous modified chitosan solution,
wherein the modified chitosan comprises an alkylated chitosan, the reaction product of an alkylated chitosan and an acidic polysaccharide, the reaction product of an alkylated chitosan and an oxidized polysaccharide, the reaction product of an alkylated chitosan, an acidic polysaccharide, and an oxidized polysaccharide, or the reaction product of an alkylated chitosan, an acidic polysaccharide, a carboxyl activating reagent, and a dehydrating reagent,
wherein the alkylated chitosan comprises an acrylated chitosan or a poly(oxyalkylene)chitosan.

28. A method for obtaining hemostasis, tissue sealing, tissue gluing, and filling tissue voids, the method comprising:
applying to one or more moist body tissue surfaces a biocompatible adherent sheet comprising a carrier sheet comprising a biocompatible polymer, wherein the carrier sheet is flexible, conformable, and of substantially uniform thickness; and a modified chitosan substantially evenly disposed on one or both surfaces of the carrier sheet,
wherein the modified chitosan comprises an alkylated chitosan, the reaction product of an alkylated chitosan and an acidic polysaccharide, the reaction product of an alkylated chitosan and an oxidized polysaccharide, the reaction product of an alkylated chitosan, an acidic polysaccharide, and an oxidized polysaccharide, or the reaction product of an alkylated chitosan, an acidic polysaccharide, a carboxyl activating reagent, and a dehydrating reagent,
wherein the alkylated chitosan comprises an acrylated chitosan or a poly(oxyalkylene)chitosan.

29. The method of claim 28, wherein the obtaining hemostasis, tissue sealing, and tissue gluing occurs during surgical interventions in a gastrointestinal system, parenchymal organs, a cardiovascular system, a thoracic system, a pulmonary system, an ear area, a nose area, a throat area, a dental area, a gynecological system, a urological system, a vascular system, a bone system, a neurological system, a lymphatic system, a dermal surface, a biliary system, or a combination thereof.

30. A biocompatible adherent sheet for application to one or more moist body tissue surfaces for obtaining hemostasis, tissue sealing, tissue gluing, and filling tissue voids, comprising:
a carrier sheet comprising a biocompatible polymer and an optional modified chitosan, wherein the carrier sheet is flexible, conformable, and of substantially uniform thickness,
wherein the modified chitosan comprises an alkylated chitosan, the reaction product of an alkylated chitosan and an acidic polysaccharide, the reaction product of an alkylated chitosan and an oxidized polysaccharide, the reaction product of an alkylated chitosan, an acidic polysaccharide, and an oxidized polysaccharide, or the reaction product of an alkylated chitosan, an acidic polysaccharide, a carboxyl activating reagent, and a dehydrating reagent,
wherein the alkylated chitosan comprises an acrylated chitosan or a poly(oxyalkylene)chitosan; and
an optional modified chitosan substantially evenly disposed on one or both surfaces of the carrier sheet.
